(19) United States
(12) Patent Application Publication
(10) Pub. No.: US 2009/0054950 A1
(43) Pub. Date: Feb. 26, 2009

(54) Method and Apparatus for Treating Incontinence

(75) Inventor: Anthony Clyde Neason Stephens, New South Wales (AU)

Correspondence Address: JONES DAY, 222 EAST 41ST ST, NEW YORK, NY, 10017, US

(73) Assignee: Continence Control Systems International Pyt Ltd., Chatswood, New South Wales (AU)

(21) Appl. No.: 11/885,503

(22) PCT Filed: Mar. 2, 2006
(86) PCT No.: PCT/AU06/00258

§ 371 Date: Sep. 2, 2008

(30) Foreign Application Priority Data
Mar. 2, 2005 (AU) 2005900957

(51) Int. Cl. A61N 1/36

(52) U.S. Cl. 607/41; 607/116; 607/138; 600/30

(57) ABSTRACT

A medical condition is treated using electrical stimulation of contractile tissue, such as a sphineter, as well as electrical stimulation of afferent nerves to illicite a neuron-modulation response. The device (1) and method is particular useful for treating urge incontinence where the tissue is a smooth muscle neo-sphineter (2) about the urethra and the nerves are in the pelvic region.

The disclosure of International patent applications numbers PCT/AU2005/001698 and PCT/AU00/00925 are herein incorporated in their entirety by reference.

### FIELD OF THE INVENTION

The present invention relates to a method and apparatus for treating a disorder by utilising neurological stimulation, and, particularly, but not exclusively, to a method and apparatus for treating incontinence.

### BACKGROUND OF THE INVENTION

There are a number of disorders in humans and animals which it is known to treat by stimulation of the nervous system (neurostimulation). In humans, these disorders include incontinence, both fecal and urinary. It is also known to use neurostimulation in the management of pain.

There are a number of known forms of urinary incontinence. Stress incontinence is usually caused by a failure of muscles around the bladder neck and urethra to maintain closure of the urinary outlet. Another form of urinary incontinence, known as urge incontinence may be caused by abnormally heightened activity of the nervous control of the bladder, producing unanticipated bladder contractions. People with urge incontinence may need to urinate frequently. Often, the urge to void cannot be voluntarily suppressed. Conversely, the absence of neural signals to trigger the awareness of fullness to void can result in overflow incontinence. Some people experience urinary incontinence that includes aspects of both stress and urge incontinence.

A number of treatments have been proposed for urinary incontinence, in particular stress incontinence.

In an earlier patent application, International Patent Application PCT/AU00/00925 (the disclosure of which is incorporated herein by reference) a method and apparatus is proposed for treating incontinence which includes the steps of forming a “neosphincter” from smooth muscle tissue taken from elsewhere in the patient's body, and wrapping the neosphincter around the urethra. An implantable stimulator provides an electrical signal to the neosphincter via two or more electrodes. The electrical signal stimulates the neosphincter to maintain tone about the urethra to reduce leaks from the bladder until the user wishes to urinate. A signal from a control device may cause the stimulator to stop providing the electrical signal to the neosphincter, to allow the neosphincter to relax and enable the individual to urinate.

This is believed to be an effective treatment for stress incontinence, and de facto may reduce the volume of any leaks due to an episode of urge incontinence while the neosphincter is stimulated. It is unlikely, however, to stop symptoms of urge incontinence should they exist, such as a desire to urinate more often than is required.

There is epidemiological evidence that patients who have stress incontinence may progress to also experience urge incontinence. Further, there is also evidence that people who have been surgically treated for symptoms of stress incontinence, may develop “de-novo” urge incontinence. As there are sensory nerves that assess bladder fullness and co-ordinate the act of urination, it is not surprising that even without treatment, a deficient sphincter function may lead to different perceptions of urge and reflex control of voiding. Additionally, if the patient has undergone surgery involving placement of a foreign body (for example a silicone artificial sphincter or a section of tape to reposition the bladder outlet) to provide a treatment for stress incontinence, it is likely that this may be provocative to the sensory pathways that provide input to bladder control and lead to the development of de-novo urge incontinence.

A technique, referred to as “neuromodulation” is known, in which low level background electrical stimulation of afferent nerve fibres (that is, the sensory nerves that bring information to the central nervous system) is used to modify the response of reflex pathways. This has been commercialised by Medtronic Inc, Minneapolis, Minn. USA for treatment of forms of urge incontinence. Medtronic propose treatment by stimulation of the sacral roots to provide background stimulation to modulate the activity of the voiding reflex neural pathways, as the nerves emerge from, and enter into the spinal cord. It has also been proposed to use microstimulators placed in various locations to stimulate sensory pathways at the periphery of the nervous system and modify reflex behaviour (US 2002/0055761, Advanced Bionics Corporation), to address symptoms of incontinence.

### SUMMARY OF THE INVENTION

In accordance with an embodiment, the present invention provides a method of treating urinary incontinence, including the step of applying an electrical signal to stimulate a sphincter to cause it to contract about the urethra, and to stimulate one or more afferent nerves to alleviate or avoid symptoms of urge incontinence.

In one embodiment, the one or more nerves are afferent nerves. Stimulation may be by way of electrical stimulation of tissue in the region of the one or more nerves.

Stimulation of the sphincter advantageously provides an effective treatment for stress incontinence and also may prevent extent of leaks which may occur as a result of urge incontinence. Electrical stimulation of one or more afferent nerves (advantageously located near the neosphincter), alleviates symptoms of urge incontinence where urge incontinence exists, or may avoid symptoms of “de-novo” urge incontinence developing, decreasing the frequency and or extent of leaks. It is believed that the novel combination of electrical stimulation of a sphincter to maintain a seal of the bladder, together with afferent nerve stimulation to alleviate symptoms of urge incontinence may prove a most effective treatment for incontinence having both stress and urge aspects.

In an embodiment, the electrical signal may include a first electrical signal for stimulating the sphincter and a second electrical signal for stimulating the one or more nerves.

In an alternative embodiment, a single electrical signal of a pre-determined pattern may be used to stimulate the sphincter and the one or more nerves. In this embodiment, the predetermined pattern may comprise one signal for effecting stimulation of the afferent nerves interspersed with a signal for stimulating the sphincter.

In an embodiment, the same electrode or electrodes may deliver the electrical signal to stimulate the sphincter and to stimulate the one or more nerves.

In an alternative embodiment, separate electrodes may deliver the electrical signal to stimulate the sphincter and to stimulate the one or more afferent nerves.

In an embodiment, the sphincter is an implanted smooth muscle neosphincter, as disclosed in the earlier International Patent Application No. PCT/AU00/00925. Branches of the pudendal nerve innervate many areas of the pelvic floor. Selective, background stimulation of the neosphincter by the electrical signal may therefore elicit a neuromodulation type affect, reducing symptoms of urgency. In one embodiment, therefore, the electrical signal is used to provide stimulation of the afferent nerves in the region of the neosphincter. One or more electrodes may be used to stimulate the one or more nerves and also to stimulate the neosphincter.

In an alternative embodiment, an electrode or electrodes for stimulating the one or more afferent nerves may be placed away from the sphincter. In one embodiment, the electrode or electrodes may be placed within the pelvic anatomy. In one embodiment, an electrode or electrodes may be arranged to provide stimulation to the External Urethral Sphincter to elicit a neuromodulation type affect from this site.

In the method of PCT/AU00/00925, the smooth muscle neosphincter is constantly stimulated to maintain tone, except, in one embodiment, during urination. Neuromodulation to reduce symptoms of urge incontinence may also generally require constant stimulation of the one or more afferent nerves.

In an embodiment, rather than constant stimulation of the one or more nerves, stimulation may be by way of a duty cycle, the nerve stimulation being on for a period and off for a period.

In an embodiment, the step of applying the electrical signal to stimulate one or more nerves may comprise initially applying the electrical signal at a relatively low level and increasing it to the required level. This “ramping up” may reduce annoying perception of the stimulation by the patient. Otherwise they may perceive this stimulation as an unpleasant tingling, for example.

In one embodiment, the stimulation of the sphincter is by a relatively low frequency signal and the stimulation of the one or more nerves is by a relatively high frequency signal.

In one embodiment, a relatively low frequency signal means from 1 to 5 Hz and a relatively high frequency signal means 5 to 200 Hz.

In an embodiment, a relatively low intensity signal is utilised for stimulation of the one or more afferent nerves and a relatively high intensity signal for stimulation of the sphincter.

In an embodiment, the method is implemented with a smooth muscle neosphincter treatment for stress incontinence, as disclosed in the above-referenced International Patent Application. A first electrical signal is utilised to stimulate the smooth muscle neosphincter, in accordance with the disclosure of the above-referenced PCT application, and a second electrical signal or signals is used to stimulate one or more afferent nerves. In an embodiment, the afferent nerves stimulated by the second electrical signal are near the EUS, or they may be elsewhere within the pelvic or lumbar region.

In the embodiment where a neosphincter is utilised in accordance with the applicant's referenced patent application, the method may include a pre-calibration step. A physician may adjust the electrical signal stimulating the neosphincter to avoid any perceived urgency symptoms, or reduce any perceived urgency symptoms. The physician may also adjust the stimulation of the one or more afferent nerves in order to optimise the effect of the stimulation.

In accordance with a second embodiment, the present invention provides, in a treatment for incontinence which includes a step of stimulating a sphincter to maintain mechanical seal of the bladder, a method of reducing or avoiding symptoms of incontinence comprising the step of providing an electrical signal to stimulate one or more afferent nerves.

In accordance with a third embodiment, the present invention provides an apparatus for treating mixed urinary incontinence in a patient, the apparatus including a stimulator device including signal generator means arranged to provide an electrical signal for stimulation of a sphincter to contract the urethra of the patient, and to stimulate one or more nerves to alleviate or avoid symptoms of urge incontinence.

In an embodiment, the signal generator is arranged to generate a single electrical signal for stimulation of the sphincter and the one or more nerves. The signal may be of a particular signal pattern, for example, interleaving a first electrical signal pattern for stimulating the sphincter with a second electrical signal pattern for stimulating the one or more nerves.

In an alternative embodiment, the signal generator may be arranged to generate separate signals for separate application to the sphincter and one or more nerves, respectively.

In an embodiment, a plurality of separate signals may be generated for a plurality of nerves or for a plurality of positions in the pelvic or lumbar anatomy. In an embodiment, these signals may have different characteristics, depending upon the effect that they are arranged to elicit.

In an embodiment, the stimulator device is arranged to be implanated within the patient's body.

In an embodiment, the apparatus includes at least one electrode for applying the electrical signal.

In an embodiment, the apparatus includes a plurality of electrodes. One or more electrodes may provide stimulation to the sphincter, and one or more other electrodes may provide stimulation to another area for stimulating the one or more nerves. In one embodiment, the one or more other electrodes may be placed elsewhere in the pelvic anatomy. In one embodiment, an electrode is arranged to be positioned to stimulate the afferent nerves in the area of the external urinary sphincter (EUS).

In an embodiment, at least a first electrode is mounted by an electrode mounting arrangement. The electrode mounting arrangement may include a mounting element having an inner surface and an outer surface. The inner surface mounts the first electrode and is arranged to be positioned proximate to the sphincter for stimulation of the sphincter. In an embodiment, a second electrode is also mounted by the electrode mounting arrangement. In an embodiment, the second electrode is mounted on the outer surface of the mounting element, for stimulating afferent nerves in the region of the sphincter. In an embodiment, one or more electrodes may be arranged to be positioned away from the first electrode elsewhere in the patient's anatomy.

In one embodiment, an electrode may be arranged to be positioned proximate to the pelvic floor in use.

In an embodiment, the signal generator means is arranged to provide a relatively high frequency signal to stimulate the nerves and a relatively low frequency signal for stimulation of the sphincter.

In an embodiment, the signal generator is arranged to provide a relatively low intensity signal to stimulate the nerves and a relatively high intensity signal to stimulate the sphincter.

In accordance with an embodiment, the signal generator means is arranged to generate the signal for stimulation of the one or more nerves as a continuous signal.

In an embodiment, the signal generator means is arranged to provide a signal for stimulating the one or more nerves in accordance with a duty cycle, being on for a period and off for a period.

In an embodiment, the signal generator means is arranged to provide the signal for stimulating the nerves by commencing with a low intensity signal and gradually increasing the signal, thereby, reducing or avoiding patient discomfort.

In an embodiment, the apparatus is arranged to provide electrical stimulation to a neosphincter such as the neosphincter disclosed in the above-referenced PCT application. In an embodiment, the neosphincter is a smooth muscle sphincter.

In an embodiment, the apparatus includes a control device. The control device may be arranged externally from the patient for patient control of the stimulator device. The control device may be adjustable to control a parameter(s) of the electrical signal. As an example, the patient may reduce the stimulation to address unwanted perception of the stimulation or, alternatively, increase stimulation to have greater inhibitory effect on the perception or frequency of urge events while the system is operative. The control device may be arranged to enable the patient to control onset and offset of stimulation.

In an embodiment, the apparatus includes a programmer device. The programmer device is arranged to adjust the parameters of the electrical signal. For example, under control of the physician, the programmer device may be used to calibrate the stimulation, for example, to assess the ability of different parameters of the electrical signal to achieve the intended clinical outcome (for example, extent of urinary leakage observed at a particular stimulus intensity). In an embodiment, the calibration may be done by the physician in response to patient feedback.

In an embodiment, the stimulator device is enclosed in a single housing. Leads may extend from the housing to the electrode(s).

In the above embodiments of the invention, the application is for treating urinary incontinence. The present invention is not limited to the treatment of urinary incontinence, but may be used to treat a number of patient disorders where stimulation of contractile tissue (such as, for example, a sphincter or smooth muscle neosphincter) in conjunction with stimulation of one or more nerves to effect a neurostimulation or neuromodulation effect, may be useful. Other applications may include faecal incontinence, where a contractile tissue may be used to facilitate closure of the colo-rectum, and neuromodulation may be used further facilitate fecal continence. Neuromodulation may also be used to reduce the perception of pain.

In accordance with a fourth aspect, the present invention provides a method of treating a disorder in a patient, comprising the steps of applying an electrical signal to stimulate contractile tissue to contract and also to effect neuromodulation by stimulation of one or more nerves.

The disorder may be fecal incontinence and the contractile tissue may be a sphincter placed to affect the colorectum or anal canal. The sphincter may be a smooth muscle sphincter.

In accordance with a fifth aspect, the present invention provides an apparatus for treating a disorder in a patient, the apparatus comprising a stimulator device including a signal generator means arranged to provide an electrical signal for stimulation of contractile tissue and also to effect neuromodulation by stimulation of one or more nerves.

In one embodiment, the disorder may be fecal incontinence and the contractile tissue may be a sphincter placed to affect the colo-rectum or anal canal. The sphincter may be a smooth muscle sphincter.

In accordance with a sixth aspect, the present invention provides an electrode arrangement for stimulating contractile tissue and one or more nerves, the electrode arrangement including at least one mounting element mounting a first electrode arranged for stimulation of the contractile tissue, and a second electrode being arranged for stimulation of the one or more nerves.

In one embodiment, the mounting element is an elongate element having inner surface and an outer surface, the first electrode is mounted on the inner surface and the second electrode is mounted on the outer surface.

In one embodiment, the electrode arrangement includes a pair of mounting elements arranged to be positioned opposite each other with the contractile tissue in between.

The electrode arrangement may include electrodes not mounted by the mounting element, positionable remote from the mounting element.

### BRIEF DESCRIPTION OF THE DRAWINGS

Features and advantages of the present invention will become apparent from the following description of embodiments thereof, by way of example only, with reference to the accompanying drawings, in which:

FIGS. 1a and 1b are diagrams of the female and male bladder anatomy, showing a site of implant of a neosphincter;

FIG. 2 is a diagram of a female bladder anatomy illustrating an implanted stimulator device in accordance with an embodiment of the present invention;

FIG. 3 is a block diagram of the stimulator of FIG. 2;

FIG. 4 is a block diagram of an apparatus in accordance with an embodiment of the present invention;

FIG. 5 is a block diagram of an apparatus in accordance with an embodiment of the present invention;

FIG. 6 shows a diagram of the bladder anatomy with a neosphincter and stimulation electrode in accordance with an embodiment of the present invention, in a position of the neosphincter;

FIG. 7 is a diagram showing a detail from FIG. 6 and illustrating operation of the embodiment of FIG. 6;

FIG. 8 is a diagram illustrating operation of a further embodiment of the present invention;

FIG. 9 is a diagram illustrating operation of yet a further embodiment of the present invention;

FIG. 10 is a cross-sectional diagram of the colorectal anatomy showing an implanted stimulator device and sphincter in accordance with a further embodiment of the present invention.

FIGS. 11a and 11b are perspective views from above and one side of electrode arrangements in accordance with embodiments of the present invention;

FIG. 12 is a diagram illustrating the electrode arrangement in accordance with an embodiment of the present invention in position about contractile tissue; and

FIG. 13 is a diagram illustrating the interleaving of the electrical stimulation in accordance with an embodiment of the present invention.

### DESCRIPTION OF PREFERRED EMBODIMENTS

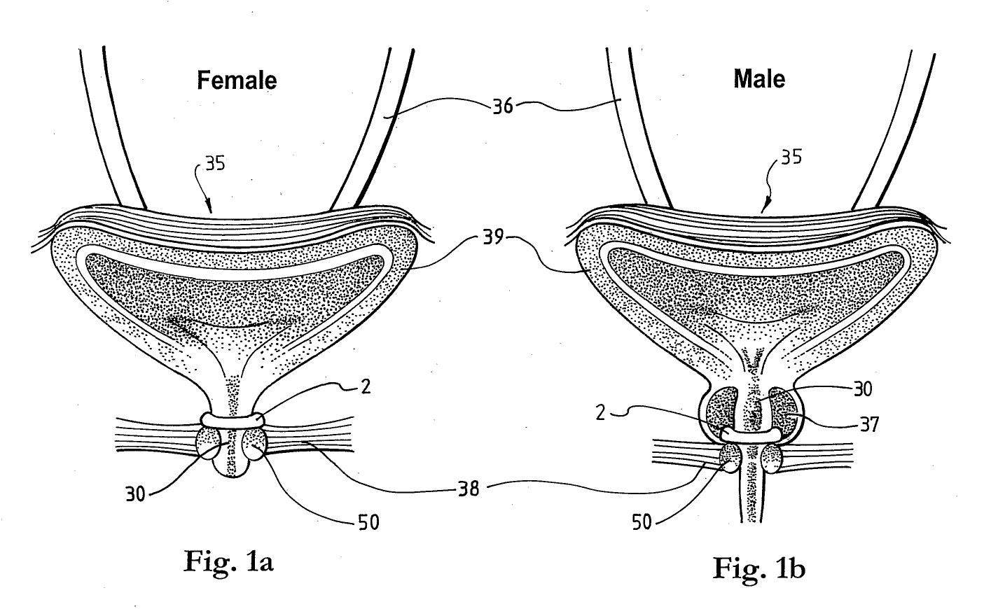

FIGS. 1a and 1b are diagrams showing aspects of the female and male urinary anatomy. The bladder in each case is designated generally by reference numeral 35. The ureters are designated by reference numeral 36. In the male anatomy the prostate gland is designated by reference numeral 37. Reference numeral 50 indicates the External Urethral Sphincter (EUS) and reference numeral 38 the pelvic floor. Reference numeral 39 indicates the Detrusor smooth muscle. The urethra in each Figure is denoted by reference numeral 30. A smooth muscle neosphincter 2 has been surgically implanted around the urethra 30 in a position close to the bladder 35. The sphincter 2 has been implanted in accordance with the disclosure of the above-referenced PCT application no. PCT/AU00/00925.

Note that the position of the neosphincter implant may be different from that shown in the diagram in some cases. It may be implanted proximal (close to the bladder) or more distal (close to the pelvic floor).

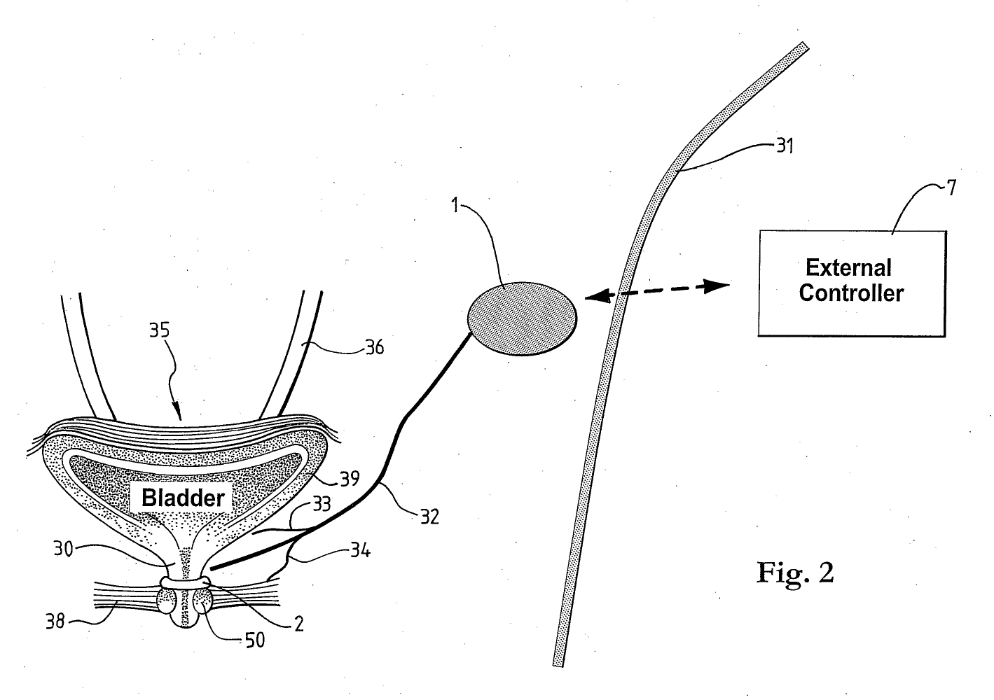

Referring to FIG. 2, which shows the female anatomy only (but it will be appreciated a similar arrangement can be transposed to the male anatomy), in accordance with an embodiment of the present invention, a stimulator device 1 has been implanted in the patient. The stimulator 1 may be implanted in any surgically convenient position, but is preferably implanted between the abdominal muscles and the skin (represented by the line designated by reference numeral 31). The stimulator 1 includes a signal generator means arranged to provide an electrical stimulation signal for stimulating the smooth muscle sphincter 2, and also to provide stimulation for afferent nerves in order to ameliorate or avoid symptoms of urge incontinence. The afferent nerves are not shown in FIG. 2, but it will be appreciated that many branches of the pudendal nerve are positioned in the region of the bladder anatomy, close to where the smooth muscle neosphincter is positioned and elsewhere in the pelvic floor. In embodiments, stimulation to the neosphincter may also stimulate the afferent nerves, in an alternative embodiment stimulation may be applied to other areas of the pelvic floor, for example in the area of theExternal Urinary Sphincter (EUS) 50. Stimulation may be applied to the Sacral Nerve roots.

In FIG. 2, conductors 32, 33, 34 are shown. Conductors 32, 33, 34 are arranged to conduct electrical stimulation signals to implanted electrodes. In another embodiment, conductors 33 and 34 may be joined by “plug-in” sockets to the conductor 32 to permit the surgeon flexibility in choosing a particular configuration suitable for the patient's condition.

In one embodiment, only a single conductor 32 may be required to provide stimulation to the smooth muscle sphincter 2 and to the afferent nerves. As discussed above, as there may be many afferent nerves in the region of the neosphincter 2, the single conductor may be sufficient to provide appropriate stimulation to affect symptoms of urge incontinence.

In another embodiment, one or more electrodes may be implanted in other parts of the pelvic anatomy. In one embodiment, electrodes may be implanted elsewhere to stimulate afferent nerves. Further leads 33 or 34 may be arranged to conduct the electrical signal to the further electrodes.

A plurality of electrodes placed in various positions may be used to provide the electrical signal (s). Each electrode may deliver a distinct electrical signal to a plurality of different sites in order to effect the required nerve stimulation.

The stimulation of afferent nerves in the pelvic anatomy is arranged to suppress symptoms of urge incontinence or avoid such symptoms developing. The symptoms of urge incontinence may be primary symptoms or symptoms developed as a result of stress incontinence problems. Further, as discussed in the preamble, implantation of the device (such as the electrode 32) has the potential to result in development of urge incontinence as it will be closely placed to sensory nerves of the External Urinary Sphincter.

The electrical stimulating signal may comprise a single signal, or two or more separate or interleaved signals. In one embodiment, a first electrical signal is responsible for stimulation of the smooth muscle sphincter 2 to maintain pressure on the urethra 30, and a second electrical signal is responsible for stimulation of the afferent nerves to ameliorate or avoid symptoms of urge incontinence. Stimulator 1 is arranged to produce all the signals with a single signal generator, although more generally, separate signal generators and even separate stimulators will still fall within the scope of the present invention.

In this embodiment, parameters of the stimulating signal(s) being produced by the stimulator 1 are variable, to enable adjustment of the stimulus, as will be discussed in more detail later.

In accordance with the system disclosed in the above-referenced PCT application, the stimulator 1 may also be arranged to produce a further electrical signal to stimulate the sphincter 2 to relax, to allow urine to flow through the urethra and enable the patient to evacuate their bladder. Instead of a further electrical signal, the stimulator 1 may be arranged to stop producing any electrical signal and it is the absence of a signal that causes the sphincter to relax.

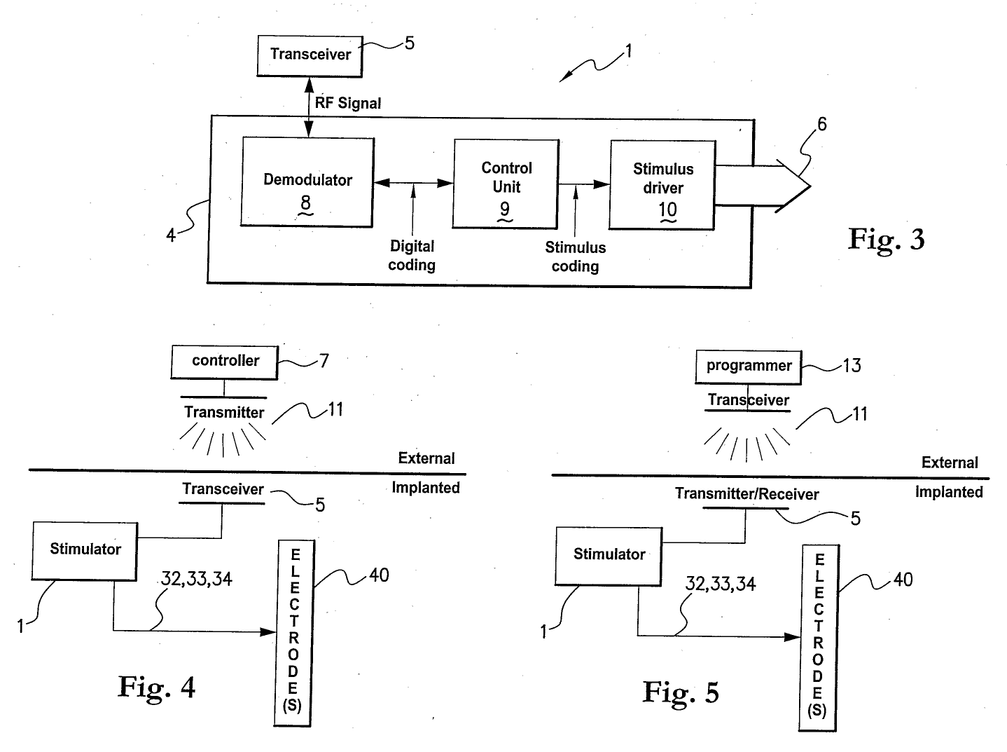

The stimulator 1 is shown in more detail in FIG. 3. In this embodiment, a signal generator means arranged to provide the electrical signal for stimulation of the sphincter and of afferent nerves is in the form of a control unit 9 and stimulus driver 10. The control unit 9 encodes the stimulus and provides a signal to the stimulus driver 10 which provides the stimulation signal at output 6. As discussed above, the output 6 may output to one or more conductors 32, 33, 34 as required and to one or more electrodes. The control unit 9 may be arranged to control the stimulus driver 10 to provide a plurality of stimulation signals e.g. one or more stimulation signals to contract the sphincter 2 and one or more stimulation signals to stimulate afferent nerves to affect symptoms of urge incontinence.

In this embodiment, the control unit 9 and stimulus driver 10 form, together with a demodulator 8, a processing unit for generating the stimulation signal(s) at output 6.

The modulator 8 is arranged to demodulate a signal received by transceiver 5. An external control unit and external programmer unit (both to be described later) are able to communicate via the transceiver with the processing unit 4 in order to control application of stimuli and/or vary the stimuli. In addition, as described in more detail later, the processing unit 4 may transmit, via control unit 9, demodulator 8 and transceiver 5, signals to the control unit or programmer unit. The transmitted signals may deliver telemetry information indicative of parameters of the stimulator, for the purposes of calibration and control.

The entire stimulator 1 (including components 4 and 5), is enclosed in a housing which includes a casing made from a bio-compatible material, such as titanium, silicone rubber or other known inert materials. The frequency of the RF signal for transmission and reception by the transceiver 5 may depend on the material of the casing of the stimulator.

FIG. 4 shows an apparatus in accordance with an embodiment of the present invention. The apparatus incorporates the implanted stimulator 1, including transceiver 5. The electrode(s) 40 is shown schematically together with cables 32, 33, 34. As discussed above, less or more than three conductors may be present, depending upon the number of electrodes present.

The apparatus also comprises an external controller 7 which includes a transmitter 11. The controller 7 is intended for operation by a patient with the stimulator implanted, for control of the stimulator 1.

The controller 7 includes means (such as a button, not shown) operable by the patient to selectively send signals to the implanted stimulator 1, for control of the stimulation signals being sent to the electrodes 40. In this embodiment, the stimulator is “fail safe”. Unless a signal is received from the controller 7, the stimulator produces a signal which maintains tone in the smooth muscle implant 2, maintaining pressure on the urethra. The stimulator also provides an electrical signal to stimulate the afferent nerves to reduce or avoid symptoms of urgency incontinence.

When the patient wishes to urinate, they actuate the controller 7 to send, via the transmitter 11, a signal to the stimulator. In response to receiving the signal, the control unit 9 operates to turn the stimulating signal off causing the sphincter to relax and allow the patient to urinate. The signal may also cause the afferent nerve stimulation to turn off. In an alternative embodiment, afferent nerve stimulation may continue or change to a different pattern of stimulation.

The controller 7 may also be arranged to provide a further signal under patient control, once the patient has finished urinating, the further signal causing stimulator 1 to resume providing the stimulation signals to the electrode(s) 40.

In “fail safe” mode, if the further signal is not produced, the stimulator will resume providing the stimulation signal to the electrodes 40 after a pre-determined period of time.

The stimulation signal 6 provided to contract the smooth muscle sphincter 2 is selected so as to provide a substantially continuous tone in the sphincter. A generally rectangular and symmetrically biphasic pulse may be suitable for this. The signal has a substantially constant current less than or equal to 30 mA, and may be in the order of 15 mA. Stimulation pulse frequency provided to sphincter 1 is in the range of 0.25 Hz to 2.5 Hz and is preferably 2 Hz. Stimulation pulse width is in the range of 0.05 m/s to 0.02 m/s and is preferably 0.15 m/s. The stimulator is current regulated, and accordingly the stimulation voltage will vary with the resistance of the muscle tissue between the electrodes. Typical values for the voltage are between 0.2 and 12 Volts. Either a current source (voltage limited) or a voltage source (current limited) stimulator may be used.

Note that it is also possible to use an asymmetric biphasic pulse, in which, for example, the first phase is shorter in duration than the second phase.

In this embodiment, the signal for stimulating the afferent nerves is a further signal provided at output 6 and may be provided to either the same electrode i.e. interleaved with the signal for stimulating the smooth muscle sphincter 2 or a separate electrode also stimulating in the sphincter area, or a separate electrode arranged to stimulate in the area of the external urethral sphincter. The further signal may provide a low-level “background” stimulation to large diameter afferent nerve fibres. A narrow width, low amplitude signal may be used to target the lower threshold, large diameter sensory fibres of the external urethral sphincter. The signal may be a long duty cycle stimulation (e.g. five minutes on, five minutes off).

Stimulation of the afferent fibres may therefore be provided in at least these two ways:

1. By the same signal that is used to stimulate the smooth muscle sphincter to contract the smooth muscle sphincter 2. In this case the signal would be arranged to provide the appropriate stimulation to maintain tone in the smooth muscle sphincter 2, as well as stimulation to reduce or avoid symptoms of urge incontinence.

2. Separate signals, one for reducing or avoiding urge incontinence and the other for stimulating the smooth muscle sphincter. The signals may be interleaved or may be provided in separate channels. In either case, the stimulation parameters can be distinct for the different purposes of (i) stimulating the smooth muscle neosphincter and (ii) stimulating the afferent nerves. The signals for stimulating the afferent nerves may be applied to other electrodes in the area of the smooth muscle sphincter 2 or in other regions in the pelvic anatomy, including the EUS.

FIG. 13 shows a diagram of an example of an interleaved-type signal. The signal 600 for stimulation of the afferent nerves has a relatively small amplitude and a higher frequency. The signal 601 for stimulating the neosphincter, has a relatively large amplitude and low frequency.

A relatively high frequency signal may be used to provide the stimulation for the afferent nerves and a relatively low frequency signal to stimulate the sphincter. The relatively high frequency signal may be greater than 5 Hz, preferably greater than 8 Hz and even more preferably 10 Hz or greater. In one embodiment, the high frequency signal is up to 100 Hz.

The low frequency signal, for stimulation of the sphincter, will usually be less than 5 Hz and may be 2 Hz or less.

A relatively low intensity signal may be used to stimulate the afferent nerves and a relatively high intensity signal for the sphincter. The low intensity signal in this embodiment is a pulse signal. The pulse signal may have 300 us or less pulse width and a 3 mA or less current, and in this embodiment 200 us or less and 1 mA current.

The high intensity sphincter stimulating signal may have pulse width greater than 300 us and a current of greater than 3 mA, and in this embodiment is 400 us or more and the current is 4 mA.

The duty cycle for the afferent nerve stimulating signal may vary. As discussed above in this embodiment it is 5 minutes on and 5 minutes off but in other embodiments may be 5 minutes or more on and 15 minutes or more off, 5 minutes or less off and 5 minutes or less on. Duty cycle and other signal characteristics may be adjusted to provide the most effective stimulation. In some embodiments, the duty cycle may be in terms of seconds (e.g. less than 10 seconds on and less than 30 seconds off).

With the afferent nerve stimulation signal, it is possible that the patient may experience some discomfort (e.g. ‘tingling’) on application of the signal. Where the signal is applied in accordance with a duty cycle, this tingling may be experienced in rhythm with the duty cycle. In one embodiment, the afferent nerve stimulating signal may be “ramped up” from a relatively low intensity to the required intensity each time it is applied. This may reduce unwanted awareness of the stimulation or patient discomfort.

The electrodes employed may incorporate an electrically conductive surface that is in contact with the neosphincter, which also activates afferent nerve fibres. In another embodiment, the electrode for the neosphincter may include one or more additional electrically conductive surfaces that are on the outer surface of the electrode, to stimulate nerve endings in the surrounding pelvic anatomy. In yet a further embodiment, the electrode may be entirely separate to the electrode used to stimulate the neosphincter, but placed conveniently in the adjacent anatomy to facilitate delivery of electrical stimulation to neuromodulate the bladder reflexes.

Referring to FIG. 11(a), an electrode arrangement 100 in accordance with an embodiment of the present invention is illustrated. The electrode arrangement 100 includes first and second electrode elements 200, 300, which in this example are in the form of elongate rectangular elements which extend from a mounting 400. Mounting 400 acts to secure the electrode elements 200, 300 at their proximal ends.

The electrode arrangement 100 includes electrodes 500, 600, in this embodiment being in the form of conductive plates which extend along the inside of the electrode elements 200, 300 opposite to each other. The electrodes 500, 600, may alternatively be in the form of a printed conductive medium printed on the inside surface of the electrode elements 500, 600.

The electrode elements 200, 300 are arranged so that tissue from a human or animal body may be received therebetween within the gap 201, so tissue is “sandwiched” between the electrode 500, 600 exposed surfaces.

In this embodiment, the electrode element 200, 300 are comprised mainly of insulating material 202 so that the outer surface (in this embodiment all surfaces apart from conductive electrodes 500 and 600) are insulated and do not conduct electricity.

In this embodiment the inner surface is also insulating material with an elongate opening in the form of a slit. The electrodes are provided at the slit.

Distal ends 203, 204 of the electrode elements 200, 300 include projecting portions which project inwardly from the electrode elements 200, 300 so that they meet each other. In operation, the ends 203, 204 may be secured together so that the electrode arrangement is firmly secured about the tissue which lies in the gap 201 between the electrode elements 200, 300.

In this embodiment, mounting 400 includes a strain relief member 205 for receiving an electrical conductor 206 within a cable 207 (the cable being insulated), the electrical conductor 206 being arranged for electrical connection between electrodes 500 and 600 and the stimulator 1. The distal ends of the electrode elements may be fixed (if required to be fixed) by a number of means, including suture holes, press studs or any other arrangement that may not require much surgical access to “lock” the electrode elements closed. Note also, it is not essential for all embodiments that the electrode elements be fixed together at their distal ends.

In embodiments, insulating parts of the electrode elements may be composed of two sheets of bio compatible material (e.g. silicone)—which acts as an insulator, and surrounds and limits the exposed surface of thin flexible platinum foils that forms the electrodes. The silicone may be reinforced with bio compatible mesh (eg a PET or PTFE-like material), so that sutures will not tear through the silicone.

In this embodiment, the electrode elements 200, 300 are arranged to flex such that the arrangement is arranged to conform, at least to some limited extent, with the profile of the received tissue and/or external tissue that they may be seated against. The electrode elements 200, 300, are arranged to flex to conform with any changes in the profile of the received tissue which may be due to electrical stimulation.

In this embodiment, the electrode elements may be sufficiently flexible so that they conform with any changes in the profile of the received tissue and also with the profile of external tissue so they do not irritate or erode the external tissue or receive tissue. This has the advantage of increasing the lifetime of the implant.

The electrode elements may be semi-flexible or in another embodiment totally flexible.

In a further embodiment, electrodes elements are not flexible (non flexible electrode elements are within the scope of the present invention).

The electrode is similar in structure and operation to the electrode disclosed in the Applicant's co-pending application, number PCT/AU2005/001698, the disclosure of which is incorporated herein by reference. In addition, a further electrode 210 is provided on the outside of at least one of the electrode elements 200. Note the yet a further electrode (not shown) may be provided on the outside of the other electrode element 300, if required. The further electrode 210 is electrically connected to the stimulator 1 via the electrical conductor 206 (note that the cable 207 may in fact house a plurality of electrical conductors if required, or a single electrical conductor may be utilised to transmit interleaved signals. Separate electrical conductors may be convenient for separate electrical signals).

FIG. 11(b) shows an alternative embodiment, same reference numerals have been used for similar components and no further description will be given of these components. In this embodiment, no further electrode is mounted on the outside of the elements 200, 300, but instead further electrodes are arranged for mounting elsewhere in the patient's anatomy, the further electrodes being designated by reference numerals 211 and 212. Electrodes 211 and 212 may be mounted remote from electrodes 500, 600, for stimulation of other areas of the pelvic anatomy. Such electrodes may comprise rounded, conductive button electrodes that may be sutured directly by the surgeon at the required location or catheter electrodes that be placed and secured between fascia or other convenient anatomy, close to the afferent nerve. Such electrodes may be connected to the electrode using an implantable connector, to provide the surgeon the convenience of using these additional electrodes at his or her convenience.

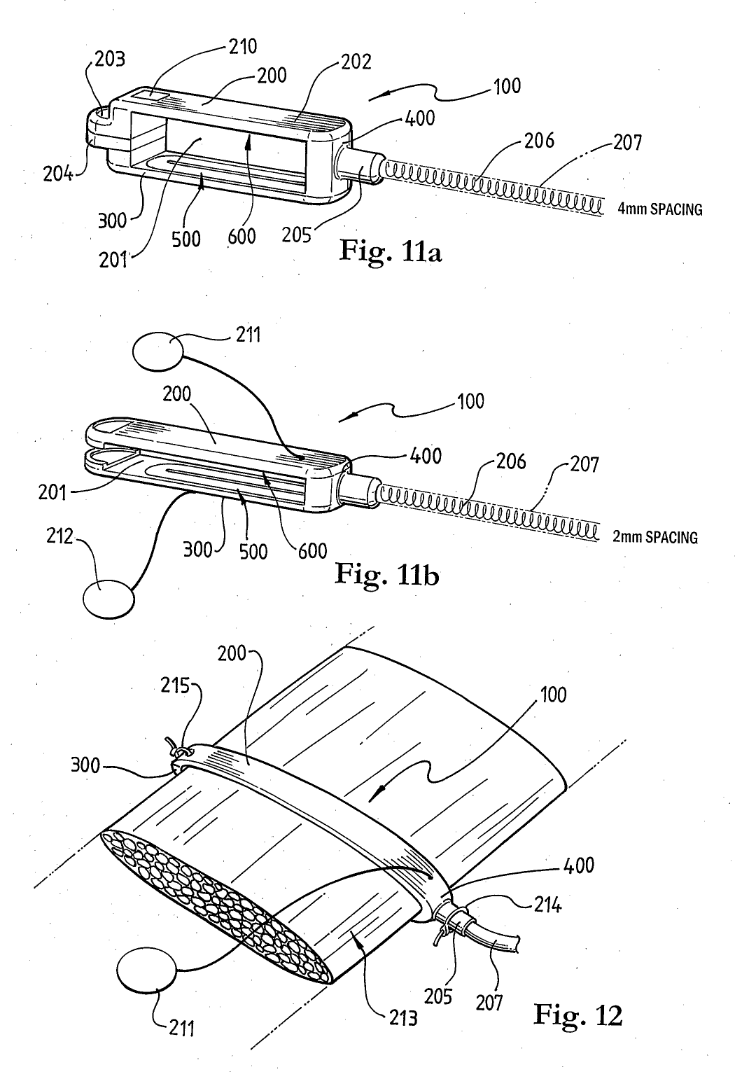

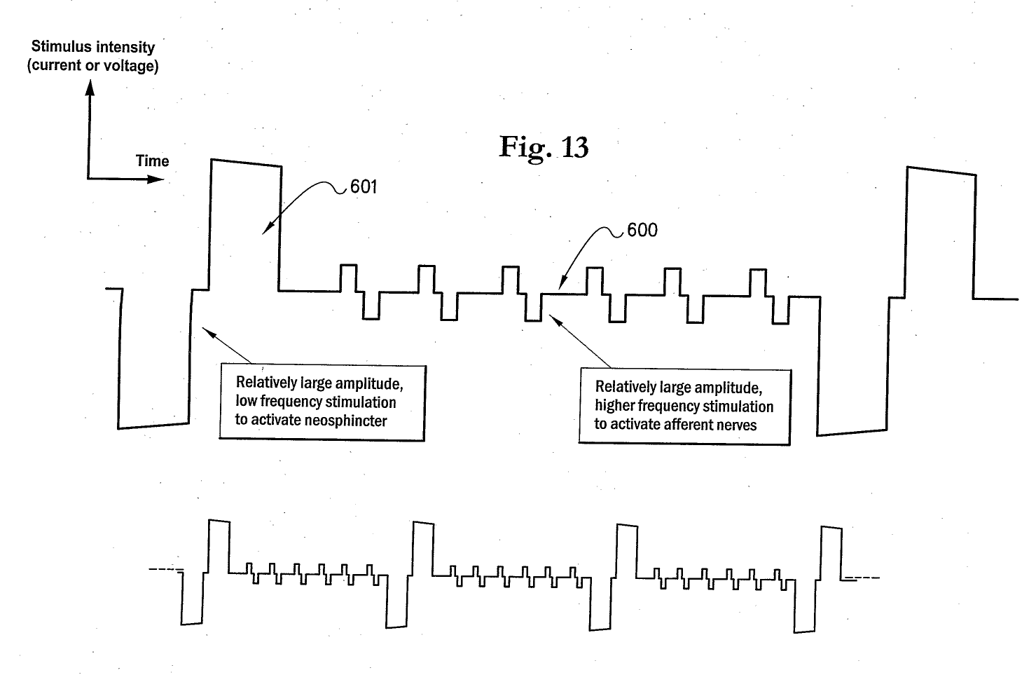

FIG. 12 schematically illustrates an electrode in accordance with an embodiment of the present invention in situ about muscle tissue 213. The same reference numerals have been used in FIG. 12 as in FIG. 11(b), to designate corresponding components.

It can be seen from FIG. 12 that the muscle tissue 213 is received between electrode elements 200, 300. The electrodes 500, 600 on the inside surfaces of the electrode elements 200, 300 therefore contact the muscle tissue 213. Electrical signals may be applied to the electrodes 500, 600 resulting in an electrical field applied across the muscle tissue 213.

Tethers (also known as sutures) 214 and 215 may be used to hold the electrode arrangement in place adjacent the tissue within the human or animal body.

In the above embodiments, the electrode arrangement 100 includes mounting elements joined at a base 400. In an alternative embodiment, the mounting elements 200, 300 may not be joined at the base but may merely be arranged to be positioned opposite each other without requiring a base. For example, they may be fixed in place in the patient e.g. sutured in place.

Regarding the FIG. 11(b) embodiment, note that there may be less or more remote electrodes 211, 212 than shown, depending upon the application and how many electrode are required for appropriate stimulation. Similarly with the embodiment of FIG. 11(a) there may be more than one electrode 210 on the outer surface of the mounting element 200, 300. Further, in another embodiment, electrodes may be included on the outer surface of the mounting element 200, 300 and also remote electrodes may be provided.

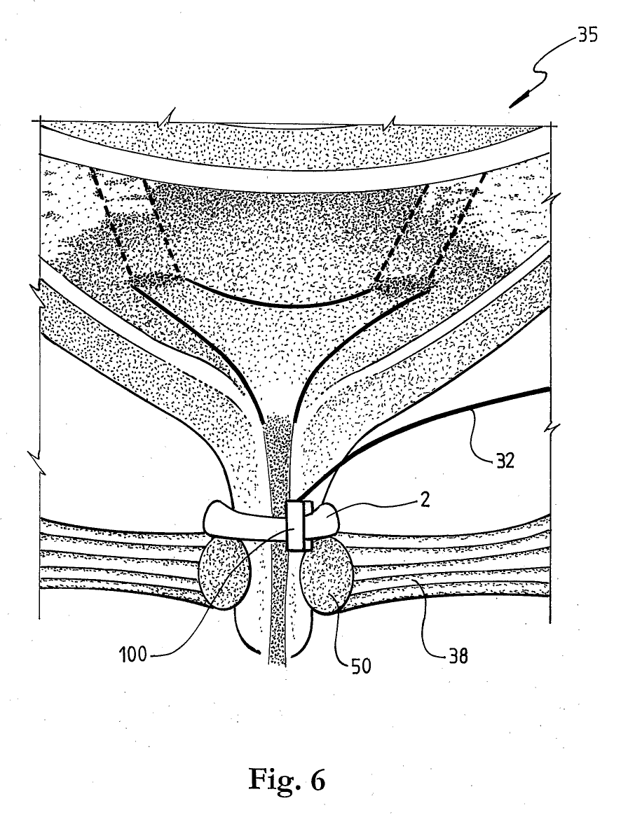

FIG. 6 illustrates a stimulation electrode 100 in place at the neosphincter 2. This electrode may be similar to the embodiment of FIG. 1.

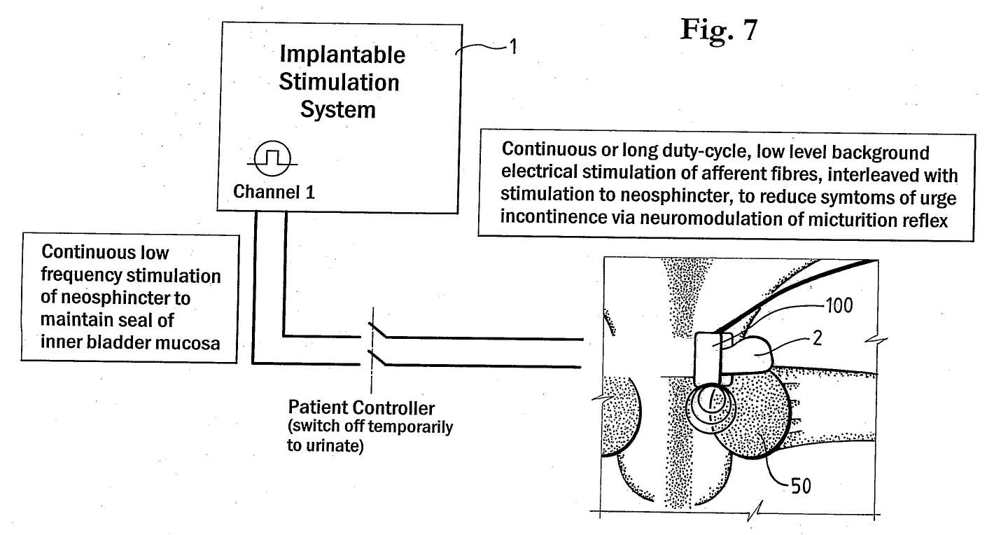

FIG. 7 schematically illustrates a system for management of mixed incontinence which utilises a stimulator 1 which provides single channel stimulation to the electrode 100 to stimulate the neosphincter 2 as well as stimulate afferent nerve fibres to reduce unnecessary symptoms of urge. The continuous or long duty cycle, low level background electrical stimulation of afferent fibres, interleaved with stimulation to the neosphincter 2, reduces symptoms of urge incontinence via neuromodulation of the micturition reflex. Background stimulation delivered to the neosphincter 2 to keep it toned may also elicit low level stimulation of sensory nerve fibres in the EUS 50, causing a neuromodulation effect, reducing symptoms of urge. The stimulus pattern delivered to the afferent nerves may be the same stimulus pattern as delivered to the neosphincter 2 or another stimulus pattern may be delivered to the afferent nerves, interleaved with the stimulation to the neosphincter.

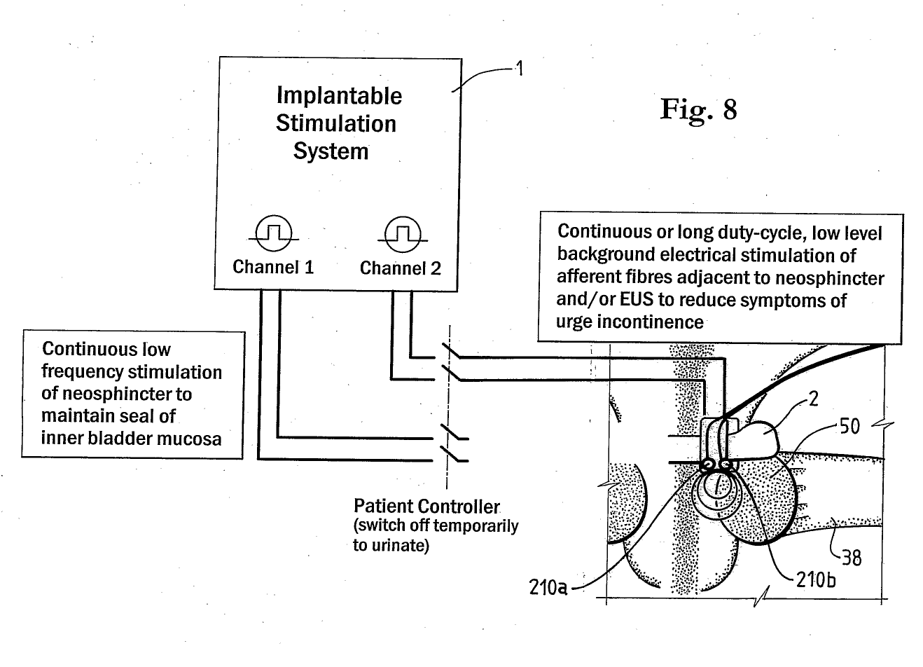

FIG. 8 illustrates schematically a two-channel implantable stimulator 1 where one channel is dedicated to stimulate the neosphincter 2 and another channel to stimulate afferent nerves to reduce the symptoms of urge incontinence. Extra conducting electrodes 210a, 210b are provided at the distal edge of the electrode 100 or on its outer surface, to activate nerve fibres near the pelvic floor 38 or EUS 50. So a separate stimulation pattern for neuromodulation can be delivered to afferent fibres in the pelvic floor or EUS from electrode sites at the distal edge or on the outer surface of the electrode 100.

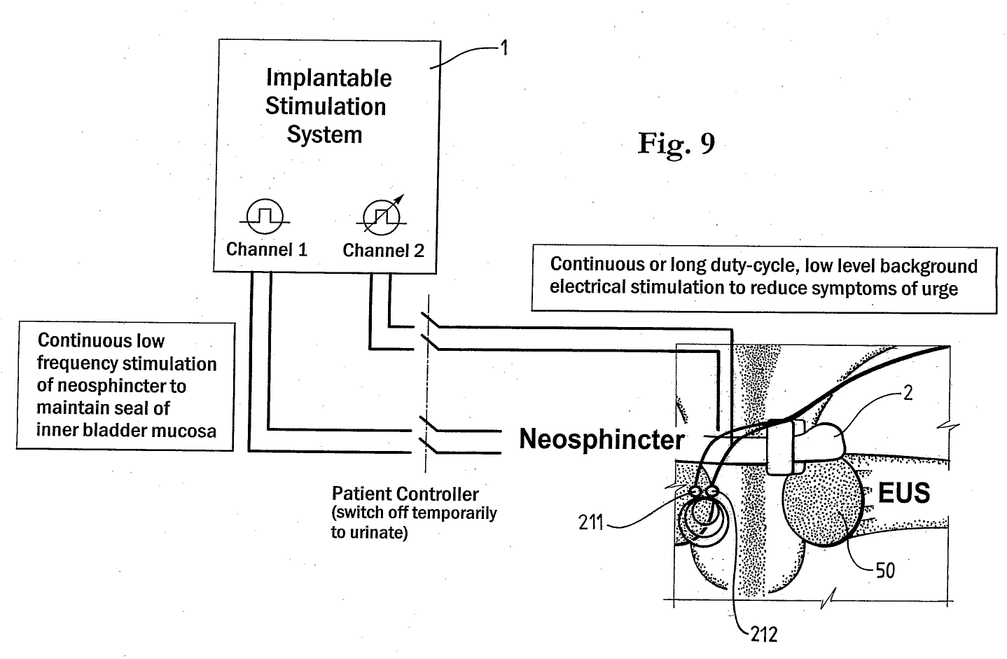

FIG. 9 illustrates a system using a two-channel implantable stimulator 1. This embodiment also illustrates an arrangement for delivering a separate stimulation pattern for neuromodulation from one or more dedicated electrodes 211, 212 that may be placed in contact with the pelvic floor or other anatomy with afferent fibres (for example, branches of the pudenal nerve).

As well as switching the stimulator 1 on and off, the controller 7 may have further patient control functions. It may, in one embodiment, include “fine” control to vary the parameters of the stimulation being applied by the stimulator 1. For example, before the patient goes to sleep, they may wish to provide a different pattern of stimulation to the afferent nerves. The controller 7 may therefore include a patient manipulator or control to enable the patient themselves to adjust the stimulation.

FIG. 5 shows an apparatus in accordance with an embodiment of the present invention, including a programmer unit 13 which may be utilised by a physician to set and adjust parameters of the implanted stimulator 1. The programmer unit 13 may include an appropriate means for communicating with the stimulator via transceiver 11, and may include a computing device. The control unit 9 is also arranged to transmit stimulator telemetry information indicative of one or more of the parameters of the stimulator 1, for detection by the programmer 13 via transceiver 11. The programmer unit 13 can therefore determine parameters of the stimulator from telemetry information and can adjust the parameters by transmitting control signals to the stimulator 1. The signal from the programmer may be able to selectively vary the output current, shape, frequency and/or pulse width or stimulation mode of the stimulation signal(s).

In operation, a physician adjusts parameters of the stimulation signal (s). The physician will note feedback from the patient as to the effect of the stimulus on bladder control, and may subsequently re-adjust the parameters until the stimulation is optimum. For example, patient perceived feedback may be used to set the maximum stimulation threshold of the smooth muscle sphincter (for example, any excess stimulation to the neosphincter may elicit and/or be perceived as an urgency event by the patient).

In the above-described embodiments, signals between the controller or programmer and the stimulator are RF signals. Other types of transmission media other than RF may be used. For example, microwave signals may be used for transmission, optical signals may be used, and in another embodiment magnetic transmission may be used.

Magnetic transmission may be used for the controller unit 7 to cause the stimulator to stop producing stimulation signals and therefore allow the patient to urinate. In this embodiment, the control unit 7 may be a simple magnet which, when passed over a magnetic receiver of the stimulator 1, results in the stimulator ceasing to provide stimulation signals for contracting the sphincter.

The apparatus of the above embodiment provides stimulation signals to a neosphincter in the form of a smooth muscle sphincter which is usually taken from elsewhere in the body and transplanted around the urethra, as taught in International Patent Application publication no. WO 01/10357. The apparatus of the present invention is not limited to providing stimulation signals to such a neosphincter. Signals may instead be provided to other anatomical features. For example, the external urinary sphincter may be stimulated to exert pressure on the urethra, in the absence of any smooth muscle neosphincter. In addition, of course, in accordance with the present invention stimulation is provided to avoid or ameliorate symptoms of urgency.

The above embodiments have been described for use mainly in applications for treating urinary incontinence. The present invention is not limited to the treatment of urinary incontinence. The present invention may be useful for any disorder where advantage may be gained by stimulating contractile tissue and at the same time stimulating one or more nerves in order to, for example, provide a neuromodulation effect, whether to decrease symptoms of urge or for example, perception of pain.

Another application where the present invention is useful is in the treatment of fecal incontinence. Signal stimulation to existing anal sphincters may be utilised in conjunction with afferent nerve stimulation. Branches of the sacral nerve may also be stimulated, for example as there are afferent nerves that also conveniently located to the anal sphincter.

In the Applicant's co-pending provisional patent application, Australian patent application number 2005905673, the disclosure of which is herein incorporated by reference, a treatment for fecal incontinence is proposed which involves stimulation of a smooth muscle sphincter wrapped about a portion of the anal canal or colo-rectal canal. In accordance with an embodiment of the present invention, neurostimulation can be applied to facilitate anal continence or treat symptoms associated with implantation of the stimulator and sphincter.

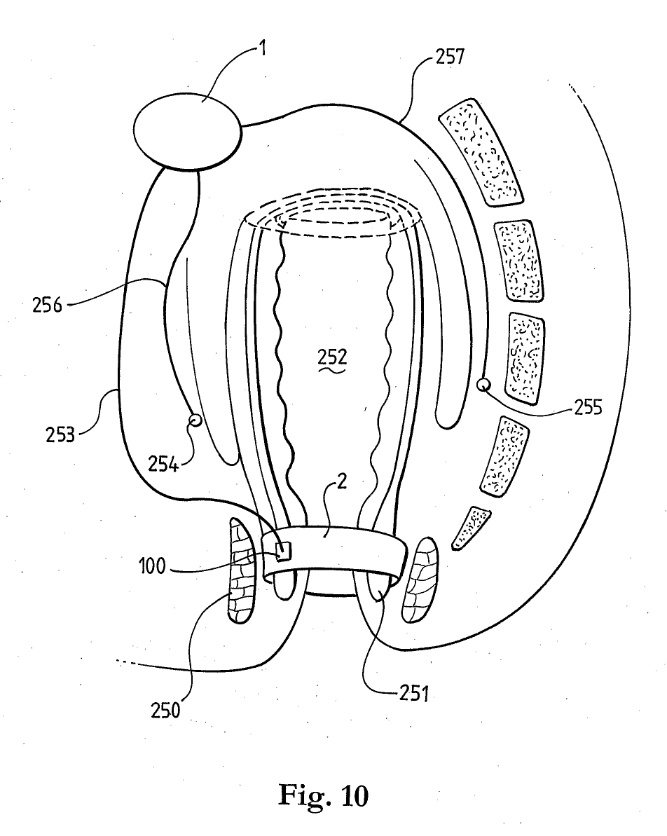

Referring to FIG. 10, a system and apparatus in accordance with an embodiment of the present invention, for treating anal incontinence, are illustrated in schematic form. The system includes an apparatus comprising an implantable stimulator 1 and a device comprising contractile tissue 2 which is arranged to be stimulated by a signal that is generated by the stimulator 1 and, in this embodiment, applied to the contractile tissue 2 via an electrode 100 conductively connected between the stimulator 1 and contractile tissue 2.

In this embodiment, the stimulator 1 may be of the same general construction as described above with reference to FIGS. 3, 4 and 5, with the signal characteristics being provided as appropriate for the anal incontinence application.

The contractile tissue 2 in this embodiment is formed into a sphincter which is implanted about the anal sphincter region, in this embodiment proximate to the anus. In FIG. 10, the external anal sphincter is designated by reference numeral 250 and the internal anal sphincter by reference numeral 251. Failure of operation of the external and/or internal anal sphincters (perhaps because of nerve damage, or other reason) have lead to anal incontinence in this patient. Stimulation of the contractile tissue sphincter 2, in operation, causes the contractile tissue 2 to contract and maintain closure of the anal-rectal canal 252, maintaining anal continence.

The stimulator 1 as discussed above includes a signal generator arranged to provide a stimulation signal for stimulating the smooth muscle sphincter 2. A lead 253 extends from the stimulator 1 to the electrode 3 at the smooth muscle sphincter 2, for providing the stimulation signal 2 to the smooth muscle sphincter 2. The stimulation signal may be a signal of frequency and amplitude determined to maintain contraction of the smooth muscle sphincter 2 to facilitate and anal continence.

The stimulator 1 may also be arranged to produce a further electrical signal to stimulate the sphincter 2 to relax, to enable the patient to defecate. As an alternative to a further electrical signal, the stimulator 1 may be arranged to stop producing any electrical signal, and it is the absence of the signal that causes the sphincter 2 to relax. In this embodiment, the stimulator 1 is arranged to have the stimulation signal varied under control of the patient by way of an external controller.

In addition to the lead 253, and electrode 100 further electrodes 254, 255 and leads 256, 257 are provided to allow background stimulation e.g. neuromodulation, to be provided to other areas of the anatomy.

In the above embodiments, power sources for the implantable stimulator will be provided in the form of batteries. These are not shown in the diagrams. The batteries may be replaceable or may be rechargeable via inductive recharging and are incorporated within the implantable stimulator.

As discussed above, the stimulator implant is preferably sealed and encased in a biologically inert material such as a bio-compatible silicone material. Metallic electrodes and leads are preferably of platinum-iridium alloy. The connecting wires are preferably insulated with a silicone coating. The implant is preferably placed between the abdominal muscle and the skin.

In the above embodiments, a single stimulation single signal generator is used to provide the electrical signal. Other embodiments may use two or more signal generators. Other embodiments may use two or more stimulators, which may be placed in different locations.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method of treating urinary incontinence, including the step of applying an electrical signal to stimulate a sphincter to cause it to contract around the urethra, and to stimulate one or more nerves to alleviate or avoid symptoms of urge incontinence.

2. A method in accordance with claim 1, wherein the electrical signal includes a first electrical signal for stimulating the sphincter and a second electrical signal for stimulating one or more nerves.

3. A method in accordance with claim 2, wherein a plurality of electrical signals are applied for stimulating a plurality of nerves.

4. A method in accordance with claim 3, wherein the plurality of signals have different signal characteristics from each other.

5. A method in accordance with claim 1, wherein the electrical signal is a single electrical signal having a predetermined pattern for stimulating the sphincter and the one or more nerves.

6. A method in accordance with claim 1, wherein the electrical signal is applied by one or more electrodes, and wherein the same electrode or electrodes may deliver the electrical signal to stimulate the sphincter and to stimulate the one or more nerves.

7. A method in accordance with claim 1, wherein a plurality of electrodes are used to apply the electrical signal, and wherein separate electrodes are utilised to deliver the electrical signal to stimulate the sphincter and the electrical signal to stimulate the one or more nerves.

8. A method in accordance with claim 1, wherein the step of applying an electrical signal includes applying an electrical signal via an electrode placed in the pelvic or lumbar anatomy.

9. A method in accordance with claim 8, wherein the step of applying an electrical signal includes applying an electrical signal via a first electrode positioned proximate the sphincter to stimulate the sphincter, and applying an electrical signal via a second electrode positioned elsewhere to stimulate the one or more nerves.

10. A method in accordance with claim 9, wherein the second electrode is positioned close to the first electrode.

11. A method in accordance with claim 9, wherein the first and second electrodes are mounted by a single electrode mounting arrangement.

12. A method in accordance with claim 11, wherein the electrode mounting arrangement includes a mounting element having an inner surface and an outer surface, the inner surface mounting the first electrode.

13. A method in accordance with claim 9, wherein the second electrode is positioned away from the first electrode, in contact with anatomy having afferent fibres.

14. A method in accordance with claim 13, wherein the second electrode is positioned proximate a branch of the pudenal nerve.

15. A method in accordance with claim 13, wherein the second electrode is positioned proximate the pelvic floor.

16. A method in accordance with claim 9, wherein there are a plurality of second electrodes.

17. A method in accordance with claim 1, wherein the sphincter is an implanted neosphincter, composed of contractile tissue.

18. A method in accordance with claim 17, wherein the neosphincter is a smooth muscle neosphincter.

19. A method in accordance with claim 17, wherein the electrical signal provides stimulation to one or more afferent nerves in the region of the neosphincter.

20. A method in accordance with claim 1, including the further step of adjusting the electrical signal to reduce or avoid any perceived urgency symptoms.

21. A method in accordance with claim 1, wherein the step of applying an electrical signal includes the step of applying a relatively high frequency electrical signal to stimulate the one or more afferent nerves and a relatively low frequency electrical signal to stimulate the sphincter.

22. A method in accordance with claim 21, wherein the relatively high frequency electrical signal is greater than 5 Hz.

23. A method in accordance with claim 22, wherein the relatively high frequency signal is 8 Hz or greater.

24. A method in accordance with claim 23, wherein the relatively high frequency signal is 10 Hz or greater.

25. A method in accordance with claim 24, wherein the relatively high frequency signal is 50 Hz or greater.

26. A method in accordance with claim 25, wherein the relatively high frequency signal is up to 100 Hz.

27. A method in accordance with claim 21, wherein the relatively low frequency signal is equal to or less than 5 Hz.

28. A method in accordance with claim 27, wherein the relatively low frequency signal is 3 Hz or less.

29. A method in accordance with claim 28, wherein the relatively low frequency signal is 1 Hz or less.

30. A method in accordance with claim 1, wherein the step of applying an electrical signal includes the steps of applying a relatively low intensity signal to stimulate the one or more afferent nerves and a relatively high intensity signal to stimulate the sphincter.

31. A method in accordance with claim 30, wherein the relatively low intensity signal is a pulse signal having a 300 us or less pulse width and a 3 mA or less current.

32. A method in accordance with claim 31, wherein the pulse width is 250 us or less and the current is 1.5 mA or less.

33. A method in accordance with claim 32, wherein the pulse width is 200 us or less and the current is 1 mA or less.

34. A method in accordance with claim 30, wherein the relatively high intensity signal is a pulse signal having a pulse width greater than 300 us and a current of greater than 2 mA.

35. A method in accordance with claim 34, wherein the pulse width is 350 us or more and the current is 3.5 mA or more.

36. A method in accordance with claim 35, wherein the pulse width is 400 us or more and the current is 4 mA or more.

37. A method in accordance with claim 1, wherein the electrical signal for stimulating one or more nerves is continuously applied.

38. A method in accordance with claim 1, wherein the electrical signal for stimulating the one or more nerves is applied in accordance with a duty cycle, the signal being on for a period and off for a period.

39. A method in accordance with claim 38, wherein the duty cycle is one minute or more less and 5 minutes or less off.

40. A method in accordance with claim 39, wherein the duty cycle is 1 minutes or more on and 5 minutes or more off.

41. A method in accordance with claim 40, wherein the duty cycle is 5 minutes or more on and 15 minutes or more off.

42. A method of reducing or avoiding symptoms of urge incontinence for use in a treatment for urinary incontinence which includes a step of stimulating a sphincter to cause it to contract the urethra, the method comprising the step of providing an electrical signal to stimulate one or more nerves.

43. An apparatus for treating mixed urinary incontinence in a patient, the apparatus including a stimulator device including signal generator means arranged to provide an electrical signal for stimulation of a sphincter to contract the urethra of the patient, and to stimulate one or more nerves to alleviate or avoid symptoms of urge incontinence.

44. An apparatus in accordance with claim 43, wherein the electrical signal includes a first electrical signal for stimulating the sphincter and a second electrical signal for stimulating the one or more nerves.

45. An apparatus in accordance with claim 44, wherein a plurality of electrical signals are applied for stimulating a plurality of nerves.

46. An apparatus in accordance with claim 45, wherein the plurality of signals have different signal characteristics from each other.

47. An apparatus in accordance with claim 43, wherein the electrical signal is a single electrical signal having a predetermined pattern for stimulating the sphincter and the one or more nerves.

48. An apparatus in accordance with claim 43, including at least one electrode which is arranged to apply the electrical signal to stimulate the sphincter and the one or more nerves.

49. An apparatus in accordance with claim 48, the apparatus including a plurality of electrodes, one or more electrodes being arranged to provide stimulation to the sphincter, and one or more other electrodes being arranged to provide stimulation to the one or more nerves.

50. An apparatus in accordance with claim 49, wherein an electrode is arranged to be positioned to stimulate an afferent nerve in the area of the external urinary sphincter (EUS).

51. An apparatus in accordance with claim 49, the apparatus including an electrode mounting arrangement mounting a first electrode for positioning proximate the sphincter and a second electrode for positioning elsewhere in the pelvic or lumbar anatomy.

52. An apparatus in accordance with claim 51, the electrode mounting arrangement including a mounting element having an inner surface and an outer surface, the inner surface being arranged to be positioned adjacent the sphincter and mounting the first electrode, and the outer surface mounting the second electrode.

53. An apparatus in accordance with claim 49, the plurality of electrodes including an electrode being arranged to be mounted proximate the sphincter and an electrode being arranged to be mounted remote from the sphincter elsewhere in the pelvic or lumbar anatomy.

54. An apparatus in accordance with claim 43, wherein the sphincter is an implanted neosphincter.

55. An apparatus in accordance with claim 54, wherein the neosphincter is a smooth muscle neosphincter.

56. An apparatus in accordance with claim 43, further including a stimulator controller which is operable by a user to adjust the stimulation.

57. An apparatus in accordance with claim 43, further including a stimulator programmer, the stimulator programmer being arranged to enable programming of control parameters of the stimulator.

58. An apparatus in accordance with claim 43, wherein the signal generator means is arranged to generate a relatively high frequency signal to stimulate the nerves and a relatively low frequency signal to stimulate the sphincter.

59. An apparatus in accordance with claim 58, wherein the relatively high frequency signal is greater than 5 Hz.

60. An apparatus in accordance with claim 59, wherein the relatively high frequency signal is 8 Hz or greater.

61. An apparatus in accordance with claim 60, wherein the relatively high frequency signal is 10 Hz or greater.

62. An apparatus in accordance with claim 61, wherein the relatively high frequency signal is 50 Hz or greater.

63. An apparatus in accordance with claim 62, wherein the relatively high frequency signal is 100 Hz or greater.

64. An apparatus in accordance with claim 58, wherein the relatively low frequency signal is 5 Hz or less.

65. An apparatus in accordance with claim 60, wherein the relatively low frequency signal is 3 Hz or less.

66. An apparatus in accordance with claim 65, wherein the relatively low frequency signal is 1 Hz or less.

67. An apparatus in accordance with claim 43, wherein the signal generator is arranged to provide a relatively low intensity signal to stimulate the nerves and a relatively high intensity signal to stimulate the sphincter.

68. An apparatus in accordance with claim 67, wherein the relatively low intensity signal is a pulse signal having a pulse duration of 300 us or less and a current of 2 mA.

69. An apparatus in accordance with claim 68, wherein the signal period is 250 us or less and the current is 1.5 mA or less.

70. An apparatus in accordance with claim 69, wherein the signal period is 200 us or less and the current is 1 mA or less.

71. An apparatus in accordance with claim 67, wherein the relatively high intensity signal is a pulse signal and the period of the signal is greater than 300 us and the current is 3 mA or greater.

72. An apparatus in accordance with claim 71, wherein the signal period is 350 us or more and the current is 3.5 mA or more.

73. An apparatus in accordance with claim 72, wherein the signal period is 400 us or more and the current is 4 mA or more.

74. An apparatus in accordance with claim 43, wherein the signal generator means is arranged to generate the signal for stimulating the afferent nerves as continuous signal.

75. An apparatus in accordance with claim 43, wherein the signal generator means is arranged to generate the afferent nerve stimulating signal in accordance with a duty cycle, the signal being on for a period and off for a period.

76. An apparatus in accordance with claim 75, wherein the on period is 10 minutes or more and the off period is 20 minutes or more.

77. An apparatus in accordance with claim 76, wherein the on period is 8 minutes or more and the off period is 18 minutes or more.

78. An apparatus in accordance with claim 77, wherein the on period is 5 minutes or more and the off period is 15 minutes or more.

79. An apparatus in accordance with claim 78, wherein the on period is less than 1 minute or less and the off period is 5 minutes or less.

80. A method of treating a disorder in a patient, comprising the steps of applying an electrical signal to stimulate contractile tissue to contract and also to effect neuromodulation by stimulation of one or more nerves.

81. A method in accordance with claim 80, wherein the electrical signal includes a first electrical signal for stimulating the contractile tissue and a second electrical signal for stimulating the one or more nerves.

82. A method in accordance with claim 81, wherein a plurality of second electrical signals are applied for stimulating a plurality of nerves.

83. A method in accordance with claim 81, wherein the plurality of signals each have different signal characteristics.

84. A method in accordance with claim 80, wherein the electrical signal is a single electrical signal having a predetermined pattern for stimulating the contractile tissue and the one or more nerves.

85. A method in accordance with claim 80, wherein the electrical signal is generated by a single signal generator.

86. A method in accordance with claim 85, wherein the signal generator means is mounted in a housing which is implanted in the patient's body.

87. A method in accordance with claim 80, wherein the electrical signal is applied by one or more electrodes, and wherein the same electrode or electrodes may deliver the electrical signal to stimulate the contractile tissue and to stimulate the one or more nerves.

88. A method in accordance with claim 80, wherein a plurality of electrodes are used to apply the electrical signal, and wherein separate electrodes are utilised to deliver the electrical signal to stimulate the contractile tissue and the electrical signal to stimulate the one or more nerves.

89. A method in accordance with claim 88, wherein the electrical signal is applied by a first electrode positioned proximate the contractile tissue and a second electrode positioned elsewhere.

90. A method in accordance with claim 89, wherein the second electrode is positioned close to the first electrode.

91. A method in accordance with claim 80, wherein the first and second electrodes are mounted by an electrode mounting arrangement.

92. A method in accordance with claim 91, wherein the electrode mounting arrangement includes a mounting element having an inner surface and an outer surface, the inner surface mounting the first electrode proximate the contractile tissue and the outer surface mounting the second electrode.

93. A method in accordance with claim 90, wherein the second electrode is positioned remote from the first electrode and in contact with anatomy bearing afferent nerve fibres.

94. A method in accordance with claim 80, wherein the disorder is fecal incontinence.

95. A method in accordance with claim 94, wherein the contractile tissue is a sphincter positioned about the colo-rectum or anal canal.

96. A method in accordance with claim 95, wherein the sphincter is a smooth muscle sphincter.

97. A method in accordance with claim 94, wherein the electrical signal is applied to stimulate one or more nerves carrying afferent signals to the sphincter muscle complex of the anal rectal barrier.

98. A method in accordance with claim 80, wherein the step of stimulating the one or more nerves include stimulating the sacral nerve.

99. A method in accordance with claim 80, wherein the stimulation of the one or more nerves is arranged for treatment of pain.

100. A method in accordance with claim 80, wherein the disorder is urinary incontinence and the contractile tissue is a sphincter mounted about the urethra.

101. A method in accordance with claim 80, wherein the contractile tissue is smooth muscle.

102. An apparatus for treating a disorder in a patient, the apparatus comprising a stimulator device including a signal generator means arranged to provide an electrical signal for stimulation of contractile tissue and also to effect neuromodulation by stimulation of one or more nerves.

103. An apparatus in accordance with claim 102, wherein the electrical signal includes a first electrical signal for stimulating the contractile tissue and a second electrical signal for stimulating the one or more nerves.

104. An apparatus in accordance with claim 103, wherein the electrical signal includes a plurality of second electrical signals arranged for stimulation of a plurality of nerves.

105. An apparatus in accordance with claim 104, wherein the plurality of second electrical signals each have different signal characteristics.

106. An apparatus in accordance with claim 102, wherein the electrical signal is a single electrical signal having a predetermined pattern for stimulating the contractile tissue and the one or more nerves.

107. An apparatus in accordance with claim 102, the signal generator means being mounted in a housing which is implantable in the patient's body.

108. An apparatus in accordance with claim 103, further including one or more electrodes, and wherein the same electrode or electrodes may be arranged to deliver the electrical signal to stimulate the contractile tissue and to stimulate the one or more nerves.

109. An apparatus in accordance with claim 102, further including a plurality of electrodes, the signal generator means being arranged to utilise separate electrodes to deliver the electrical signal to stimulate the contractile tissue and the electrical signal to stimulate the one or more nerves.

110. An apparatus in accordance with claim 109, wherein a first electrode is arranged to be positioned proximate the contractile tissue and a second electrode is arranged to be positioned elsewhere.

111. An apparatus in accordance with claim 110, wherein the second electrode is arranged to be positioned close to the first electrode.

112. An apparatus in accordance with claim 111, further including an electrode mounting arrangement mounting the first and second electrodes.

113. An apparatus in accordance with claim 112, wherein the electrode mounting arrangement includes a mounting element having an inner surface and an outer surface, the inner surface mounting the first electrode for positioning proximate the contractile tissue, and the outer surface mounting the second electrode.

114. An apparatus in accordance with claim 110, wherein the second electrode is arranged to be positioned remote from the first electrode and in contact with anatomy bearing afferent nerve fibres.

115. An apparatus in accordance with claim 102, wherein the disorder is fecal incontinence.

116. An apparatus in accordance with claim 115, wherein the contractile tissue is a sphincter positioned about the colo-rectum or anal canal.

117. An apparatus in accordance with claim 116, wherein the sphincter is a smooth muscle sphincter.

118. An apparatus in accordance with claim 115, arranged to apply the electrical signal to stimulate one or more nerves carrying afferent signals to the sphincter muscle complex of the anal-rectal barrier.

119. An apparatus in accordance with claim 112, the signal generator means being arranged to provide the electrical signal for stimulating the sacral nerve.

120. An apparatus in accordance with claim 102, wherein the stimulation of the one or more nerves is arranged for treatment to reduce the perception of pain.

121. An apparatus in accordance with claim 102, wherein the disorder is urinary incontinence and the contractile tissue is a sphincter mounted about the urethra.

122. An apparatus in accordance with claim 121, wherein the contractile tissue is smooth muscle.

123. An apparatus in accordance with claim 43, further include the sphincter.

124. An apparatus in accordance with claim 102, further including the contractile tissue.

125. A method of surgically implanting a stimulator device in accordance with claim 43, comprising the steps of implanting the apparatus in the patient's body so that it is positioned to provide stimulation to the contractile tissue and the one or more afferent nerves.

126. A method of surgically implanting an apparatus in accordance with claim 102, comprising the steps of implanting the stimulator device in the patient's body so that it is positioned to provide stimulation to the contractile tissue and the one or more nerves.

127. An electrode arrangement for stimulating contractile tissue and one or more nerves, the electrode arrangement including at least one mounting element mounting a first electrode arranged for stimulating the contractile tissue, and a second electrode being arranged to be mounted to stimulate the one or more nerves.

128. An electrode arrangement in accordance with claim 127, wherein the second electrode is also mounted on the mounting element.

129. An electrode arrangement in accordance with claim 128, wherein the mounting element has an inner face and an outer face, and the first electrode is mounted on the inner face and the second electrode is mounted on the outer face.

130. An electrode arrangement in accordance with claim 127, wherein the second electrode is arranged to be mounted remote from the first electrode, elsewhere in the pelvic anatomy.

131. An electrode arrangement in accordance with claim 127, wherein there are a plurality of second electrodes.

132. An electrode arrangement in accordance with claim 127, wherein the are a plurality of first electrodes.

133. An electrode arrangement in accordance with claim 127, wherein the mounting element has a length dimension and a width dimension, the length dimension is greater of the width dimension, so that the mounting element is of an elongate form.

134. An electrode arrangement in accordance with claim 127, comprising a further mounting element, in operation the mounting element and further mounting element arranged to be positioned opposite to each other with the contractile tissue in between.

135. An electrode arrangement in accordance with claim 134, the mounting element and further mounting element being mounted at proximate ends thereto to a base, from which they extend in respect to each other so as to form a gap between them for receiving the contractile tissue.

136. An electrode element in accordance with claim 135, wherein the gap is open on all sides apart from when the mounting elements are joined at the base, so the arrangement may be placed over the tissue from one end, in an analogous manner to a peg.

137. A method of surgically implanting an electrode arrangement in accordance with claim 127, so that the electrode arrangement is positioned to provide stimulation to contractile tissue and the one or more nerves.
